(19) United States
(12) Patent Application Publication
(10) Pub. No.: US 2004/0234579 A1
(43) Pub. Date: Nov. 25, 2004

(54) Dietary supplements and methods of preparing and administering dietary supplements

(75) Inventor: Mark D. Finke, Scottsdale, AZ (US)

Correspondence Address: MICHAEL BEST & FRIEDRICH, LLP, 100 E WISCONSIN AVENUE, MILWAUKEE, WI, 53202, US

(73) Assignee: Mark D. Finke, Inc., Scottsdale, AZ

(21) Appl. No.: 10/443,588

(22) Filed: May 22, 2003

(51) Int. Cl. A61K 35/78

(52) U.S. Cl. 424/442; 424/725

(57) ABSTRACT

A chewable or non-chewable, palatable and shelf stable dietary supplement for animals including a carrier matrix formed of a natural substance and an effective amount of a medicament intermixed with the carrier matrix. Methods for administering a medicament to an animal may include forming a slurry from a natural substance; mixing an effective amount of a medicament with the slurry to form a mixture; pouring the mixture into a mold; freezing the mixture to form a frozen mixture; drying the frozen mixture to form a freeze-dried dietary supplement; and administering the dietary supplement to an animal. Methods for preparing a dietary supplement may include providing a natural substance to form a carrier matrix for the medicament; slurrifying the natural substance to form a slurry; mixing an effective amount of the medicament with the slurry to form a mixture; pouring the mixture into a mold; freezing the mixture; and removing moisture from the mixture to form a shelf-stable dietary supplement.

### BACKGROUND OF THE INVENTION

Existing dietary supplements incorporate pastes, tablets, gelatin capsules or soft treats to administer nutrients, nutraceuticals, medicines or other medicaments to animals, including mammals and birds.

A majority of the existing supplements for delivering most medicaments to animals are chewable or non-chewable tablets, which are prepared by pressing a variety of ingredients together under pressure. While accurate dosing is possible by administering the existing chewable and non-chewable tablets, the taste and texture of the tablets are not palatable to most mammals and birds, so administration is difficult. In addition, the additives used for the manufacturing of these tablets are usually unpalatable and objectionable. Likewise, gelatin capsules are sometimes used to administer medicaments that cannot be pressed into a tablet, such as fat or fatty acid supplements. Gelatin capsules may provide accurate dosing but are also not palatable to most mammals and birds, so administration is difficult. Therefore, to orally administer a medicament to a mammal or bird in the form of a tablet or a gelatin capsule, the tablet or gelatin capsule may need to be forced down the throat of the mammal or bird. Alternatively, the tablet or gelatin capsule may be disguised in a variety of foods (cheese or peanut butter) in an effort to increase palatability and acceptability. This method is generally not very reliable, especially for smaller mammals, cats and birds.

Soft moist treats have also been used to deliver medicaments to animals (particularly to dog and cats). However, the soft moist treats generally require sugars to enhance palatability and artificial preservatives to maintain shelf stability. In addition, unless properly packaged, the soft moist treats can dry out and become hard, brittle and unpalatable. Furthermore, preparing many of the existing soft moist treats involves the use of heat and since many medicaments are heat sensitive, this can denature or reduce the function of the medicament.

Other existing supplements include liquids and pastes. For example, existing horse wormers are administered by using a syringe (without the needle) to force the liquid or paste into the horse's mouth. The syringe is then emptied directly into the back of the horse's mouth. This method is both difficult to administer and often times results in the horse spitting out the liquid or paste.

### SUMMARY OF THE INVENTION

The present invention is generally directed to a chewable (or non-chewable), palatable and shelf stable dietary supplement for animals comprising a carrier matrix formed of a natural substance and an effective amount of a medicament intermixed with the carrier matrix.

The present invention may further comprise a method of administering a medicament to an animal, the method comprising forming a slurry comprising a natural substance; mixing an effective amount of a medicament with the slurry to form a mixture; pouring the mixture into a mold; freezing the mixture to form a frozen mixture; drying the frozen mixture to form a freeze-dried dietary supplement comprising the medicament; and administering the dietary supplement to an animal.

The present invention may further comprise a method of preparing a dietary supplement comprising a medicament, the method comprising providing a natural substance to form a carrier matrix for the medicament; slurrifying the natural substance to form a slurry; mixing an effective amount of the medicament with the slurry to form a mixture; pouring the mixture into a mold; freezing the mixture; and removing moisture from the mixture to form a shelf-stable dietary supplement.

### DETAILED DESCRIPTION

The present invention is directed to a dietary supplement for use in administering a variety of medicaments to animals (including, but not limited to, mammals and birds) in a palatable form. This dietary supplement comprises a carrier matrix formed of a natural substance and at least one medicament to be administered. The dietary supplement may not require the use of additional binding or structural agents, such as gums, gelatins, sugars, humectants, starches, polymeric materials, or the like. This invention is further directed to a method of administering the dietary supplement to an animal and a method of preparing the dietary supplement.

As used herein and in the appended claims, the term “medicament” refers to any substance administered to deliver a therapy including, without limitation, at least one of a nutrient, nutraceutical, medicine, prebiotic, probiotic, enzyme and combinations thereof. A medicament may include a synthetic compound, a plant-based compound, an animal-based compound, a derivative substance, a material that contains a medicament thereof, and combinations thereof. For example, a medicament may include flax seeds as a source of n-3 and n-6 fatty acids, chicory root as a source of inulin (a prebiotic), and bovine trachea or chicken keel as a source of chondroprotective agents. Additionally, a variety of forms of the medicament can be used to improve stability including cross-linked (vitamin A), polyphosphates (vitamin C) and encapsulated forms.

As used herein and in the appended claims, the term “nutrient” refers to any substance which helps support life. Examples of nutrients may include, but are not limited to, vitamins, minerals, protein, amino acids, fats, fatty acids, and combinations thereof.

As used herein and in the appended claims, the term “vitamin” refers to an organic nutrient required in small quantities for a variety of biochemical functions which cannot be synthesized by the body and must be supplied by the diet. Examples of vitamins may include, but are not limited to, vitamin A, vitamin D, Vitamin E, Vitamin K, Vitamin C, thiamin, riboflavin, pantothenate/pantothenic acid, niacin, pyridoxine, folate/folic acid, biotin, vitamin B₁₂, choline, and combinations thereof.

As used herein and in the appended claims, the term “mineral” refers to an inorganic nutrient required for a variety of biochemical functions which cannot be synthesized by the body and must be supplied by the diet. Examples of minerals may include, but are not limited to, calcium, phosphorus, magnesium, sodium, potassium, chloride, zinc, iron, copper manganese, selenium, iodine, chromium and combinations thereof.

As used herein and in the appended claims, the term “fatty acid” refers to a hydrocarbon chain ranging from 2 to 24 carbons or more with a carboxylic acid group on one end and a methyl group on the opposite end. Examples of fatty acids may include, but are not limited to, supplements of omega-3 and omega-6 fatty acids, such as fish oil, canola oil, borage seed oil, flax oil, evening primrose oil, sunflower oil, safflower oil, corn oil, soybean oil and combinations thereof.

As used herein and in the appended claims, the term “nutraceutical” refers to any substance that is produced in purified or extracted form and administered orally to animals to provide agents required for normal body structure and function and administered with the intent of improving the health and well-being of animals. Hand et al., 2000 (Hand MS, Thatcher CD, Remillard RL, and Roudebush P. Small Animal Clinical Nutrition 4^{th} Edition, Walsworth Publishing Marceline, Missouri 2000. pp. 1192+) describes and defines “nutraceutical” and is incorporated herein by reference. Examples of nutraceuticals may include, but are not limited to, flavenoids, carotenoids, fatty acids, taurine, carnitine, glucosamine, chondroitin, conjugated linoleic acid, and combinations thereof. Adebowale et al., 2002 (Adebowale A, Du J, Liang Z, Leslie JL, Eddington ND The bioavailability and pharmacokinetics of glucosamine hydrochloride and low molecular weight chondroitin sulfate after single and multiple dosages to beagle dogs. Biopharm Drug Dispos 2002 23:217-225) and Hand et al., 2000 describe various examples of nutraceuticals and are incorporated herein by reference.

As used herein and in the appended claims, the term “medicine” refers to any compound or substance which is designed to help mitigate or cure a specific disease or medical condition. Examples of medicines may include, but are not limited to, antihelmetics, such as ivermectin, moxidectin, fenbendazole, oxibendazole, oxifendazole, pyrantel pamoate, pyrantel tartrate and praziquantel (for horses and other species); aspirin; phenylbutazone; mineral oil or petroleum jelly (for hairball prevention in cats); and combinations thereof. Hand et al., 2000 describes how mineral oil and/or petroleum jelly may be used for hairball prevention in cats and is incorporated herein by reference.

As used herein and in the appended claims, the term “probiotic” refers to a preparation containing viable, defined microorganisms in sufficient number which alter the microflora in a compartment of the host and yield beneficial health effects in the host. Examples of probiotics may include, but are not limited to, Lactobacillus species, Bifidobacterium species, Enterococcus species, Lactococcus species, Streptococcus thermophilus, Bacillus cereus and combinations thereof. Holzapfel et al., 2001 (Holzapfel WH, Haberer P, Geisen R, Bjorkroth J, and Schillinger U. Taxonomy and important features of probiotic microorganisms in food and nutrition. Amer. J. Clin Nutr. 2001 73:365S-373S) defines and describes various probiotics and is incorporated herein by reference.

As used herein and in the appended claims, the term “prebiotic” refers to a non-digestible food or feed ingredient that beneficially affects the host by selectively stimulating the growth and/or activity of one or a limited number of bacteria in the colon Flickinger et al., 2003 (Flickinger E A, Loo J V and Fahey C F. Nutritional responses to the presence of inulin on olifofructose in the diets of domesticated animals: A review. Critical reviews in Food Science and Nutrition 2003 43:19-60) defines and describes various prebiotics and is incorporated herein by reference. Examples of prebiotics may include, but are not limited to, at least one of inulin, dried chicory root, frucooligosaccharide, mannaoligosaccharide, soybean oligosaccharide, xylooligosaccharide, and combinations thereof.

As used herein and in the appended claims, the term “enzyme” refers to a protein which functions to catalyze specific chemical reactions. For example, an enzyme may be administered to an animal to aid in food digestion, especially for dogs and cats with pancreatic insufficiency. Hand et al., 2000 describes how enzymes may be administered to animals to aid in food digestion and is incorporated herein by reference. Examples of enzymes may include, but are not limited to proteases, lipases, amylases, cellulases, and lactases.

As used herein and in the appended claims, the term “carrier matrix” refers to a material that provides shape, structure and support for a medicament that is to be administered. The carrier matrix may be formed of one or more natural substances.

As used herein and in the appended claims, the term “natural substance” refers to a substance which occurs naturally and does not require synthetic fabrication. A natural substance may include an edible plant-based or animal-based substance which can be emulsified and dried to form a particle of any desired weight or dimension. Examples of natural substances may include, but are not limited to, fruits, vegetables, skeletal tissue, organ meats, cartilaginous tissue, connective tissue, and combinations thereof.

Examples of “fruits” may include, but are not limited to, at least one of apples, apricots, bananas, blackberries, blueberries, cantaloupes, cherries, cranberries, figs, grapes, kiwis, mangos, papayas, peaches, pears, plums, pomegranates, strawberries, and combinations thereof.

Examples of “vegetables” may include but are not limited to, at least one of carrots, corn, peas, potatoes, pumpkins, squashes, yams, and combinations thereof.

As used herein and in the appended claims, the term “skeletal tissue” refers to osseous structures and skeletal muscle tissue. Examples of skeletal tissue may include, but are not limited to, the skeletal tissue of a mammal, the skeletal tissue of a bird, the skeletal tissue of a fish, and combinations thereof.

As used herein and in the appended claims, the term “organ meat” refers to any tissue (including, without limitation, smooth muscle tissue, vasculature, and combinations thereof) associated with any organ (including, without limitation, liver, heart, spleen, kidney, and combinations thereof) of at least one of a mammal, bird and/or fish.

As used herein and in the appended claims, the term “connective tissue” refers to any part of a mammal, bird and/or fish that comprises a source of a chondroprotective agent. Examples of connective tissues may include, but are not limited to, bovine or porcine trachea, chicken or turkey keel, and combinations thereof.

As used herein and in the appended claims, the term “cartilaginous tissue” refers to a tissue that comprises cartilage. Examples of cartilaginous tissues may include, but are not limited to, trachea, ear, nose, shark cartilage, and combinations thereof.

As used herein and in the appended claims, “chondroprotective agent” refers to compounds which counter arthritic degenerative processes and encourage normalization of the synovial fluid and cartilage matrix. Examples of chondroprotective agents may include, but are not limited to, various forms or salts of glucosamine, chondroitin, hyaluronic acid, and combinations thereof. For example, glucosamine may be administered in the form of glucosamine hydrochloride and glucosamine sulfate, and chondroitin may be administered in the form of chondroitin sulfate.

As used herein and in the appended claims, “additive” refers to any edible element and/or compound that is added to the dietary supplement either singularly or in combination to enhance at least one of the stability, texture, density, flavor, color and/or shelf life of the dietary supplement. Example of additives may include, but are not limited to, sugar, corn syrup, molasses, gum, humectant, starch, natural color, artificial color, natural flavor, artificial flavor, natural preservative, artificial preservative, stabilizer, emulsifier, and combinations thereof.

As used herein and in the appended claims, the term “mammal” refers to any warm blooded vertebrate of the class Mammalia that feeds it's young with milk from the mammary gland, that has the body more or less covered with hair, and that (except for the monotremes) brings forth living young. Examples of mammals that may receive the dietary supplement of the present invention may include, but are not limited to, small pet mammals, companion mammals and larger pet mammals. Examples of small pet mammals may include, but are not limited to, mice, rats, gerbils, hamsters, guinea pigs, rabbits, or ferrets. Examples of companion mammals may include but are not limited to cats or dogs. Examples of larger pet mammals may include but are not limited to, burros, mules, or horses.

As used herein and in the appended claims, the term “bird” refers to any warm blooded vertebrate of the class Aves having a body covered with feathers and forelimbs modified into wings. Examples of birds that may receive the dietary supplement of the present invention may include, but are not limited to, parakeets, cockatiels, lovebirds, lorikeets, conures, amazons, African grey parrots, eclectus, cockatoos or macaws.

As used herein and in the appended claims, the term “palatable” refers to a substance or material that is agreeable or pleasing to the sense of taste.

As used herein and in the appended claims, the term “shelf stable” refers to a product that can be stored at room temperature (i.e., has a shelf life) for a period of up to about 1 year, particularly, for a period of up to about 2 years, and more particularly, for a period of up to about 3 years without deteriorating, significantly altering chemical composition, significantly altering physical appearance, reducing efficacy, or showing any other sign of reduced life or function. Specifically, a shelf stable dietary supplement having no additives, according to the present invention, may comprise less than 10 wt % water, particularly, less than 7 wt % water, and more particularly, less than 4 wt % water.

As used herein and in the appended claims, the term “effective amount” refers to an amount of a medicament to be administered to an animal to yield the desired result in the animal.

As used herein and in the appended claims, the terms “dosing regimen” or “dosage” refers to an effective amount of a medicament to be administered to an animal at a specific frequency for a specific animal body weight. Table 1 below provides examples of dosing regimens for a variety of medicaments administered to a variety of animals.

As used herein and in the appended claims, the term “slurrify” refers to the act of forming a slurry of a given material or substance. Slurrifying may include, but is not limited to, emulsifying, stabilizing, grinding, blending, homogenizing, and combinations thereof. Slurrifying a mixture does not necessarily refer to homogenizing a mixture, but may include homogenizing.

As used herein and in the appended claims, the term “emulsifier” refers to any compound or agent that aids in forming an emulsion from one or more constituents. Examples of emulsifiers may include, but are not limited to, monoglycerides and diglycerides, polysorbates, sodium lauryl sulfate, sucrose esters and lecithin.

As used herein and in the appended claims, the term “stabilizer” refers to any compound or agent that aids in stabilizing an emulsion from one or more constituents. Examples of stabilizers may include, but are not limited to, various gums including xanthan, guar, karaya, locust bean, methylcellulose, carboxymethylcellulose, carageenan, agar and pectin.

Generally, the present invention is directed to a dietary supplement that includes a freeze-dried or air-dried natural substance as a carrier matrix for orally administering a variety of medicaments to animals, particularly, to mammals and birds. The dietary supplement may be formed by slurrifying a natural substance, mixing an effective amount of a medicament with the slurrified natural substance to form a mixture, freezing the mixture to form a frozen mixture, and drying the frozen mixture to form the supplement. The dietary supplement can have a variety of shapes, sizes, textures, densities, flavors, colors, and the like.

The success of a dietary supplement to administer a particular medicament to an animal according to a specified dosing regimen is at least partially dependent on the palatability of the dietary supplement. The dietary supplement of the present invention is effective in administering a variety of medicaments to animals, particularly, to mammals and birds at least partially because of the palatability of the supplement.

In preparing the dietary supplement, the natural substance is slurrified using any one of a number of available techniques including standard slurrifying equipment (e.g., crushers, grinders, impactors, pulverizers, blenders, food processors, emulsifiers, mortar and pestle, etc.) and/or chemicals (e.g. emulsifiers, enzymes, etc.). The particle size of the slurrified natural substance will vary depending upon the natural substance used and the desired characteristics of the finished product. For example, a smoothly textured product requires a finer grind, while a coarsely textured product requires a coarser grind. The particle size of the particles may vary depending on the desired slurry consistency. The slurry may be pasteurized to destroy objectionable organisms/contaminants without significantly altering the chemistry of the slurry. Other additives may be added to the slurry, such as those described above, to enhance various characteristics of the dietary supplement.

The medicament to be administered is added to the slurry, and the resulting mixture is blended or mixed using any of a variety of blenders or mixers known to those of ordinary skill in the art. The medicament may be added to the slurry to form a mixture in a weight percent of medicament to slurry of less than approximately 20 wt % in some embodiments, less than approximately 10 wt % in other embodiments, and less than approximately 1 wt % in still other embodiments. The medicament may be added to the slurry to form a mixture in a weight percent of medicament to slurry of greater than approximately 0.2 wt % in some embodiments of the present invention, greater than approximately 0.5 wt % in other embodiments, and greater than approximately 1 wt % in still other embodiments. The amount of medicament added to the slurry to form a mixture may vary depending on a variety of factors, including the animal to receive the dietary supplement, the size of the supplement, the medicament or combination of medicaments to be administered, the natural substance used to form the slurry, the solubility of the medicament in the slurry, and the presence of additives in the mixture.

The mixture may be blended or mixed either at room temperature or at a temperature below room temperature to either maintain the freshness of the carrier matrix or to improve the stability of the medicament. For example, when the carrier matrix is fresh skeletal tissue, liver or other organ meats, a mixing temperature of less than room temperature may prevent spoilage of the carrier matrix. Mixing times will vary depending upon the carrier matrix, the amount of medicament and the solubility of the medicament in the carrier matrix. If the medicament is not readily soluble in the slurry, an emulsifier, such as lecithin, may be added. An emulsifier may be added in a weight percent of emulsifier to mixture of greater than approximately 0.1 wt %, particularly, greater than approximately 0.5 wt %, and more particularly, greater than approximately 0.8 wt %. An emulsifier may be added in a weight percent of emulsifier to mixture of less than approximately 1.0 wt %, particularly, less than approximately 0.8 wt %, and more particularly, less than approximately 0.5 wt %. The medicament may comprise more than one medicament, which may be mixed together to form a dry blend, suspension and/or solution prior to adding the medicaments to the slurry. Alternatively, when more than one medicament is used, each medicament may be mixed with the slurry successively or simultaneously.

The mixture may be poured into a mold that is appropriately shaped and sized for an animal. In other words, the mold produces a dietary supplement having an effective amount of medicament in an appropriate size for an animal. The mold may comprise a flexible polymeric material and/or a more rigid metal. Flexible plastic molds (e.g., polyethylene, polyurethane molds) are generally less expensive than metal molds and provide facile removal of molded products. The mold may comprise an open-ended tray having multiple recesses for receiving slurry and may further comprise a lid or covering.

The mold recesses may have a variety of shapes including, without limitation, hemispherical molds with a radius of greater than approximately 0.2 cm, particularly, greater than approximately 0.3 cm, and more particularly, greater than approximately 0.5 cm. The mold recesses may have hemispherical molds with a radius of less than approximately 6.0 cm, particularly, less than approximately 5.0 cm, and more particularly, less than approximately 4.0 cm. Alternatively, the mold recesses may have a rectangular box-like shape with dimensions of greater than approximately 0.5×0.5×0.5 cm, particularly, greater than approximately 0.8 cm×0.8 cm×0.8 cm, and more particularly, greater than approximately 1.0×1.0×1.0 cm. The mold recesses may have a rectangular box-like shape with dimensions of less than approximately 7.5×7.5×5.0 cm, particularly, less than approximately 6.0×6.0×3.0 cm, and more particularly, less than approximately 5.0×5.0×2.5 cm. The exact size and shape used in each situation will depend on the species for which the supplement is intended, the amount of medicament to be delivered, the need for additional compounds such as stabilizers and emulsifiers, and the natural substance to be used to form the carrier matrix.

As mentioned above, the mold shape and size depends on the animal receiving the supplement and the effective amount of medicament to be administered. A given dietary supplement may be formed of a variety of shapes and sizes to provide a variety of dosages for a variety of animal species, particularly, mammal and bird species. For example, a given combination of at least one natural substance and at least one medicament may be prepared in hemispherical molds having radii ranging from about 0.5 cm to about 3.0 cm, particularly, ranging from about 0.5 cm to about 1.0 cm for birds, small mammals, and cats; ranging from about 1.0 cm to about 2.0 cm for dogs; and ranging from 1.5 cm to about 3.0 cm for burros, mules and horses. Alternatively, the mold to form the dietary supplement may include at least one ridge to form a score in the dietary supplement and to provide a facile dosing mechanism. The scored dietary supplement may be cut along the score to a variety of sizes to allow proper dosing for a variety of animal body weights. For example, providing a dietary supplement that comprises a score or is available in a variety of sizes allows consumers to administer an accurate dosage of the medicament to be administered to dogs weighing from approximately 3 lbs (or in kg) to approximately 200 lbs (or in kg), horses weighing from approximately 200 lbs (or in kg) to approximately 2000 lbs (or in kg), etc.

Once the mixture has been poured into a mold, either at room temperature or at a temperature below room temperature to either maintain the freshness of the carrier matrix or to improve the stability of the medicament, the mixture is frozen. The mixture should be frozen at a temperature below which any water present in the mixture exists only in the solid phase, i.e., the eutectic point of the mixture. Accordingly, the freezing temperature used to freeze the mixture will vary depending on the carrier matrix, the medicament and the presence of any additives. For example, the addition of salts and sugars will depress the freezing point of the mixture, thereby requiring colder temperatures to freeze the water in the mixture.

The frozen mixture is then dried via at least one of evaporation, lyophilization, freeze-drying, critical-point drying, and combinations thereof. The frozen mixture can be dried in the mold and subsequently removed from the mold, or alternatively, the frozen mixture can be removed from the mold and subsequently dried using any of the above-mentioned moisture removal techniques, as known to those of ordinary skill in the art and described in Schwartzberg, 2000 (Schwartzberg H. Freeze Drying. In F J Francis ed. Wiley Encyclopedia of Food Science and Technology Vol 2, John Wiley & Sons, Inc. New York 2000, pp. 1106-1112), which is incorporated herein by reference. While any of the above-mentioned methods can be used to remove excess moisture from the mixture, excellent results were obtained using lyophilization. The process parameters of the lyophilization process (e.g., pressures, primary drying times, secondary drying times and shelf and product temperatures) will vary depending on the carrier matrix, the medicament and the presence of any additives, but will be readily ascertainable by those of ordinary skill in the art. One generic lyophilization example is provided below in Example 20. The parameters may be controlled to prevent product collapse, excessive shrinkage or charring. Lyophilization may result in at least one of low thermal damage, minimal loss of volatile flavors and colors, minimal loss of heat liable ingredients, minimal product shrinkage, a long shelf life if suitably packaged, and minimal loss of biological activity (i.e., for enzymes and probiotics), as described in Schwartzberg, 2000. Drying may produce a supplement that is almost completely dried, i.e., a supplement having less than about 10 wt % of moisture, particularly, less than about 7 wt %, and more particularly, less than about 4 wt %. Alternatively, partially drying the frozen mixture may result in a supplement having less than about 28 wt % of moisture, particularly, less than about 25 wt %, and more particularly, less than about 20 wt %. Partially drying the frozen mixture may result in a supplement having greater than approximately 8 wt % moisture, particularly, greater than approximately 10 wt %, and more particularly, greater than approximately 12 wt %. By not removing all the moisture from the mixture, the resulting dietary supplement may have a different texture than one that is completely dried. A partially-dried dietary supplement (e.g., a supplement comprising more than 12 wt % moisture) may require the addition of one or more additives, such as those described above to enhance a variety of characteristics of the dietary supplement, particularly, shelf stability. The additives may be added at any point in the process. That is, additives may be added to the slurry prior to the addition of the medicament, to the mixture after the addition of the medicament, or to the supplement subsequent to the drying process.

The dried dietary supplement is formed of a particular natural substance having a particular particle size and weight and comprises a particular dosage of the medicament to be administered. By varying the size of the dietary supplement and the amount of medicament added to the slurry, a variety of final concentrations of medicament may be achieved. Table 1 below shows exemplary dosages for administering a variety of medicaments (including vitamins, minerals, fatty acids, wormers, medicines and other nutrients/nutraceuticals) to a variety of animals (including horses, mules, burros, dogs, cats, ferrets, small mammals and birds). The dosages expressed in Table 1 are meant to provide examples of dosages for a variety of medicaments and animals for the present invention, but the present invention is not limited to these dosages, medicaments or animals. For some of the medicament-animal combinations in Table 1, more than one range of dosages is expressed to further exemplify medicament dosages that may be administered using the present invention.

TABLE 1
Exemplary Dosages of Medicaments

| | Horses, | | | | | |
|---|---|---|---|---|---|---|
| | Mules & | | | | Small | |
| Medicament | Burros | Dogs | Cats | Ferrets | Mammals | Birds |
| Vitamins | | | | | | |
| Vitamin A | 7.5-600 | 20-400; | 100-5,000 | 50-2,500 | 5-100 | 10-400 |
| | IU/kg | 50-200 | IU/cat/day | IU/ferret/ | IU/kg | IU/bird/day |
| | BW/day | IU/kg | | day | BW/day | |
| | | BW/day | | | | |
| Vitamin E | 0.4-9.0 | 1.0-10.0; | 1-25 | 0.5-12.5 | 0.1-2.0 | 0.25-5.0 |
| | IU/kg/BW/ | 1.0-5.0 | IU/cat/day | IU/ferret/ | IU/kg | IU/bird/day |
| | day | IU/kg | | day | BW/day | |
| | | BW/day | | | | |
| Biotin | 0.03-0.15; | | | | | |
| | 0.05-0.12 | | | | | |
| | mg/kg | | | | | |
| | BW/day | | | | | |
| Minerals | | | | | | |
| Calcium | 2-40 | 10-320; | 50-400 | 25-200 | 10-100 | 3-75 |
| | mg/kg/ | 20-200 | mg/cat/day | mg/ferret/ | mg/kg | mg/bird |
| | BW/day | mg/kg/ | | day | BW/day | day |
| | | BW/day | | | | |
| Zinc | 0.07-2.7 | 0.5-5.0; | 1-10 | 0.5-5.0 | 0.1-1.0 | 0.1-2.5 |
| | mg/kg | 0.5-2.0 | mg/cat/day | mg/ferret/ | mg/kg | mg/bird/ |
| | BW/day | mg/kg | | day | BW/day | day |
| | | BW/day | | | | |
| Fatty Acids | | | | | | |
| Omega-6 | | 25-500; | 100-2,000 | 50-1,000 | | |
| | | 50-400 | mg/cat/day | mg/ferret/ | | |
| | | mg/kg | | day | | |
| | | BW/day | | | | |
| Omega-3 | | 10-250; | 40-1,000 | 20-500 | | |
| | | 50-200 | mg/cat/day | mg/ferret/ | | |
| | | mg/kg | | day | | |
| | | BW/day | | | | |
| Wormers | | | | | | |
| Ivermectin | 0.2 mg/kg | | | | | |
| | BW | | | | | |
| Oxibendazole | 10-15 | | | | | |
| | mg/kg BW | | | | | |
| Pyrantel | 6.6-13.2 | 5-10 mg/kg | | | | |
| | mg/kg BW | BW | | | | |
| Medicines | | | | | | |
| Aspirin | | 5-15 | 20-40 | | | |
| | | mg/kg | mg/cat/ | | | |
| | | BW/twice | every 2-3 | | | |
| | | a day | days | | | |
| Phenylbutazone | 2-6; | | | | | |
| | 4 | | | | | |
| | mg/kg/ | | | | | |
| | BW/day | | | | | |
| Mineral | | | 50-5,000 | 20-1,000 | 20-200 | |
| Oil/Petroleum | | | mg/cat/day | mg/ferret/ | mg/rabbit/ | |
| Jelly | | | | day | day | |
| Other | | | | | | |
| Nutrients/ | | | | | | |
| Nutraceuticals | | | | | | |
| Glucosamine | i) 7.2-14.4; | i) 10-60; | i) 125-250 | | | |
| | 10.8 | 20-55 | mg/cat/day | | | |
| i) Treatment | mg/kg BW/ | mg/kg BW/ | | | | |
| ii) Maintenance | twice a day | twice a day | | | | |
| | ii) 3.6-7.2; | ii) 10-60; | ii) 125-250 | | | |
| | 3.6 | 20-55 | mg/cat/day | | | |
| | mg/kg BW/ | mg/kg | or every | | | |
| | twice a day | BW/day | other day | | | |
| Chondroitin | i) 2.4-4.8; | i) 8-48; | i) 100-200 | | | |
| | 3.6 | 16-45 | mg/cat/day | | | |
| i) Treatment | mg/kg | mg/kg | | | | |
| ii) Maintenance | BW/twice | BW/twice | | | | |
| | a day | a day | | | | |
| | ii) 1.2-2.4; | ii) 8-48; | ii) 100-200 | | | |
| | 1.2 | 16-45 | mg/cat/day | | | |
| | mg/kg | mg/kg | or every | | | |
| | BW/twice | BW/day | other day | | | |
| | a day | | | | | |
| Carnitine | | i) 50-100 | | | | |
| | | mg/kg | | | | |
| i) Treatment of | | BW/3 | | | | |
| cardiomyopathy | | times a day | | | | |
| ii) Supplement | | | | | | |
| | | ii) 1-10 | ii) 1-5 | | | |
| | | mg/dog/ | mg/cat/day | | | |
| | | day | | | | |
| Taurine | | i) 500- | i) 250-500 | | | |
| | | 1,000 | mg/cat/day | | | |
| i) Treatment of | | mg/dog/3 | | | | |
| cardiomyopathy | | times a day | ii) 10-100 | | | |
| ii) Supplement | | | mg/cat/day | | | |
| Prebiotics | | 25-1,000; | 250-2,500; | 125- | 75-1,750; | 25-2,500 |
| | | 40-500 | 500-2,000 | 1500; | mg/kg | mg/bird/ |
| | | mg/kg | mg/cat/day | 250-1,000 | BW/day | day |
| | | BW/day | | mg/ferret/ | | |
| | | | | day | | |

### EXAMPLE 1

Beef liver was emulsified into a slurry to form a carrier matrix for glucosamine hydrochloride, chondroitin sulfate and vitamin C. A dry blend containing 79.41 wt % glucosamine hydrochloride, 15.82 wt % chondroitin sulfate and 4.77 wt % vitamin C (70 wt % coated ascorbic acid) was formed by homogeneously mixing the constituents. The dry blend was added to the slurry of emulsified beef liver in a weight percent of dry blend to emulsified beef liver of approximately 14.2 wt % and mixed for a time sufficient to ensure uniform dispersion of the dry blend. The resulting mixture was poured into rectangularly-shaped molds and measuring (length×width×height) 1.95 cm×1.95 cm×1.36 cm, each mold forming an individual supplement. The mixture was then frozen. The frozen supplements were removed from the molds and lyophilized to remove moisture from the supplements. The dried supplements each weighed 1.7 g and contained 500 mg glucosamine hydrochloride, 100 mg of chondroitin sulfate and 20 mg vitamin C, and contained less than about 4 wt % moisture and, hence, were shelf stable. A palatability study was performed which employed 20 dogs for 2 days. The dosage of glucosamine hydrochloride in this study was 500 mg/dog/day, the dosage of chondroitin sulfate in this study was 100 mg/dog/day, and the dosage of vitamin C in this study was 20 mg/dog/day, i.e., dosages typical for medium to large breed dogs fed once or twice daily. The dogs were offered this product versus a current leading selling tableted glucosamine supplement (8 IN 1 EXCEL-brand glucosamine pet supplement) in a controlled feeding trial. In the trial, the dogs were offered a single unit of each of the two supplements, and the dog's first choice was determined. The procedure was repeated the second day of the trial with the order of the two supplements reversed (i.e., the supplement presented to the dog on the left side for day one was presented on the right side on day two). The dogs preferred the liver-based supplement of the present invention over the 8 IN 1 EXCEL-brand glucosamine pet supplement in a ratio of 2.1 to 1 (27 to 13 dogs—using first taste as a measurement).

### EXAMPLE 2

Beef liver was emulsified into a slurry to form a carrier matrix for a vitamin supplement (comprising vitamin A, vitamin D, vitamin E, vitamin C, thiamin, riboflavin, pantothenic acid, niacin, pyridoxine, biotin, folic acid and vitamin B₁₂) and a mineral supplement (comprising calcium, phosphorus, magnesium, potassium, chloride, zinc, iron, manganese, copper, iodine and selenium). A dry blend containing 66.67% mineral supplement and 33.33% vitamin supplement was formed by homogeneously mixing the constituents. The dry blend was added to a slurry of emulsified beef liver in a weight percent of dry blend to emulsified beef liver of approximately 7.50 wt % and mixed for a time sufficient to ensure uniform dispersion of the dry blend. The resulting mixture was poured into rectangularly-shaped molds measuring (length×width×height) 1.95 cm×1.95 cm×1.36 cm, each mold forming an individual supplement. The mixture was then frozen. The frozen supplements were removed from the molds and lyophilized to remove moisture from the supplements. The dried supplements each weighed 1.6 g, contained sufficient vitamins and minerals to meet approximately 25% of the requirements of these vitamins and minerals for a 35 lb dog when fed once a day and contained less than about 4 wt % moisture and, hence, were shelf stable. When dogs (20 dogs for 2 days) were offered this product versus a current leading selling tableted vitamin-mineral supplement (8 IN 1 EXCEL-brand daily multi-vitamin) in a controlled feeding trial, as described in Example 1, the freeze-dried supplements of the present invention were preferred 1.9 to 1 (26 to 14 dogs—using first taste as a measurement) over the supplement 8 IN 1 EXCEL-brand daily multi-vitamin.

### EXAMPLE 3

Fresh apples were emulsified in a slurry to form a carrier matrix for ivermectin a prophylactic worming medication commonly used for horses. Ivermectin was added to the slurry of emulsified apples in a weight percent of dry ivermectin to emulsified apples of approximately 0.672 wt % and mixed for a time sufficient to ensure uniform dispersion of the ivermectin. The resulting mixture was poured into rectangularly-shaped molds measuring (length×width×height) 4.00 cm×1.50 cm×3.00 cm, each mold forming an individual supplement. The resulting mixture made 90 supplements, each weighing approximately 17 g. The mixture was then frozen and lyophilized to remove moisture from the supplements while in the molds. The dried supplements each weighed 3 g and contained 113 mg of ivermectin, an effective amount equivalent to current horse wormers on the market. Since the supplements contained less than about 4 wt % moisture, they were shelf stable. This supplement would be used to eliminate most adult parasites and many of their larval stages in horses weighing from 900-1200 lbs and typically would be administered every four to eight weeks. It was readily accepted and consumed by horses.

### EXAMPLE 4

Fresh carrots were emulsified in a slurry to form a carrier matrix for ivermectin a prophylactic worming medication commonly used for horses. Ivermectin was added to the slurry of emulsified carrots in a weight percent of ivermectin to emulsified carrots of approximately 0.626 wt % and mixed for a time sufficient to ensure uniform dispersion of the ivermectin to form a mixture. The resulting mixture was poured into rectangularly-shaped molds measuring (length×width×height) 4.00 cm×1.50 cm×3.00 cm, each mold forming an individual supplement. The resulting mixture made 18 supplements, each weighing approximately 17 g. The mixture was then frozen and lyophilized following the process and process parameters set forth in Example 3. The dried supplements each weighed 2.5 g and contained 113 mg of ivermectin, an effective amount equivalent to current horse wormers on the market. Since the supplements contained less than about 4 wt % moisture, they were shelf stable. This supplement would be used to eliminate most adult parasites and many of their larval stages in horses weighing from 900-1200 lbs and typically would be administered every four to eight weeks.

### EXAMPLE 5

Fresh apples were emulsified in a slurry to form a carrier matrix for pyrantel pamoate a prophylactic worming medication commonly used for horses. Pyrantel pamoate was added to the slurry of emulsified apples in a weight percent of pyrantel pamoate to emulsified apples of approximately 3.51 wt % and mixed for a time sufficient to ensure uniform dispersion of the pyrantel pamoate. The resulting mixture was poured into round molds measuring (diameter×height) 4.35 cm×2.45 cm, each mold forming an individual supplement. The resulting mixture made 55 supplements, each weighing approximately 32 g. The mixture was then frozen and lyophilized following the process and process parameters set forth in Example 3. The dried supplements each weighed 8 g and contained 3,600 mg of pyrantel pamoate, an effective amount equivalent to current horse wormers on the market. Since they contained less than about 4 wt % moisture, they were shelf stable. The supplement would be used to eliminate large stronglyes, pinworms and roundworms in horses weighing from 900-1200 lbs and typically would be administered every four to eight weeks.

### EXAMPLE 6

Fresh apples were emulsified into a slurry to form a carrier matrix for glucosamine hydrochloride, chondroitin sulfate and vitamin C. A dry blend containing 70.64 wt % glucosamine hydrochloride, 25.33 wt % chondroitin sulfate and 5.81 wt % vitamin C (70 wt % coated ascorbic acid) was formed by homogeneously mixing the constituents. The dry blend was added to the slurry of emulsified fresh apples in a weight percent of dry blend to emulsified beef liver of approximately 8.21 wt % and mixed for a time sufficient to ensure uniform dispersion of the dry blend. The resulting mixture was poured into round molds measuring (diameter×height) 4.35 cm×2.45 cm, each mold forming an individual supplement. The resulting mixture made 54 supplements, each weighing approximately 31 g. The mixture was then frozen and lyophilized following the process and process parameters set forth in Example 3. The dried supplements each weighed 7 g and contained 1,800 mg glucosamine hydrochloride, 600 mg of chondroitin sulfate and 148 mg vitamin C. Since the supplements contained less than about 4 wt % moisture, they were shelf stable. The finished product would be recommended for use as a chondroprotective agent for horses. Typical usage for horses, mules and burros would initially involve administering 3-5 supplements both in the morning and evening to be gradually reduced to a maintenance level of 1-2 supplements both in the morning and evening over the course of 6-10 weeks depending upon the response of the animal.

### EXAMPLE 7

Fresh carrots were emulsified into a slurry to form a carrier matrix for glucosamine hydrochloride, chondroitin sulfate and vitamin C. A dry blend containing 70.64 wt % glucosamine hydrochloride, 25.33 wt % chondroitin sulfate and 5.81 wt % vitamin C (70 wt % coated ascorbic acid) was formed by homogeneously mixing the constituents. The dry blend was added to the slurry of emulsified fresh apples in a weight percent of dry blend to emulsified beef liver of approximately 7.28 wt % and mixed for a time sufficient to ensure uniform dispersion of the dry blend. The resulting mixture was poured into round molds measuring (diameter×height) 4.35 cm×2.45 cm, each mold forming an individual supplement. The mixture was then frozen and lyophilized following the process and process parameters set forth in Example 3. The dried supplements each weighed 6 g and contained 1,800 mg glucosamine hydrochloride, 600 mg of chondroitin sulfate and 148 mg vitamin C. Since the supplements contained less than about 4 wt % moisture, they were shelf stable. The finished product would be recommended for use as a chondroprotective agent for horses. Typical usage for horses, mules and burros would initially involve administering 3-5 supplements both in the morning and evening, depending on animal weight, to be gradually reduced to a maintenance level of 1-2 supplements both in the morning and evening over the course of 6-10 weeks depending upon the response of the animal.

### EXAMPLE 8

A dietary supplement according to the present invention, comprising a carrier matrix formed of emulsified apple and/or carrot for administering oxibendazole to horses as a treatment for equine parasites (large strongyles, pinworms, roundworms and threadworms) when used at dosages of 10-15 mg/kg body weight every four to eight weeks.

### EXAMPLE 9

A dietary supplement according to the present invention, comprising a carrier matrix formed of emulsified skeletal tissue and/or internal organs of mammals, birds and/or fish for administering pyrantel (e.g., pyrantel pamoate) to dogs as a treatment for worms when used at a dosage of 5 mg/kg body weight every three weeks until parasites and/or their eggs are no longer present in the stools.

### EXAMPLE 10

A dietary supplement, according to the present invention, comprising a carrier matrix formed of emulsified skeletal tissue and/or internal organs of mammals, birds and/or fish for administering mineral oil and/or petroleum jelly to cats or ferrets. The recommended dosage would be in the range of 20-5,000 mg/animal/day. The dietary supplement would be suitable as a treatment for the prevention of hairballs in cats when administered weekly, or to treat cats with hairballs when used daily for up to seven consecutive days.

### EXAMPLE 11

A dietary supplement, according to the present invention, comprising a carrier matrix formed of emulsified skeletal tissue and/or internal organs of mammals, birds and/or fish for administering camitine and/or taurine to dogs and cats. The dietary supplement would be suitable as a treatment for dogs and cats with dialated cardiomyopathy or myocardial carnitine deficiency. For cats, recommended dosages for taurine would be 250-500 mg/cat/day while for dogs dosages of 500-1000 mg/dog three times a day are recommended. For carnitine, the recommended dosage is 50-100 mg carnitine/dog three times a day.

### EXAMPLE 12

A dietary supplement, according to the present invention, comprising a carrier matrix formed of emulsified skeletal tissue and/or internal organs of mammals, birds and/or fish for administering carnitine and/or conjugated linoleic acid to dogs and/or cats. The dietary dosage for carnitine would be 1-10 mg/dog/day or 1-5 mg/cat/day. The dietary supplement would be suitable to help control weight and body fat in dogs and cats.

### EXAMPLE 13

A dietary supplement, according to the present invention, comprising a carrier matrix formed of emulsified skeletal tissue and/or internal organs of mammals, birds and/or fish for administering probiotics and/or prebiotics to dogs, cats and/or ferrets. The dietary supplement would be suitable to help modify the microflora in the gastrointestinal tract, thereby improving gastrointestinal health and reducing fecal odors of dogs and cats. Flickinger et al., 2003 describes using probiotics and/or prebiotics to modify the microflora in the gastrointestinal tract of animals and is incorporated herein by reference. The recommended dosage for prebiotics for dogs, cats and ferrets would range from 25-1,000 mg/kg/day. The recommended dosage for probiotics would range from 1×10⁵-1×10¹⁰ colony forming units/animal/day depending upon the animal species and the species of bacteria in the probiotic.

### EXAMPLE 14

A dietary supplement, according to the present invention, comprising to a carrier matrix formed of emulsified skeletal tissue and/or internal organs of mammals, birds and/or fish to administer Yucca schidigera to dogs and/or cats. The dietary supplement would be suitable to help reduce fecal odors of dogs and cats when used at a dosage of 2-10 mg/kg body weight/day. Lowe and Kershaw, 1997 (Lowe J A, and Kershaw S J. The ameliorating effect of Yucca schidigera extract on canine and feline faecal aroma. Research in Veterinary Science 1997 63:66-66) describes using Yucca schidigera to help reduce fecal odors of dogs and cats and is incorporated herein by reference.

### EXAMPLE 15

A dietary supplement, according to the present invention, comprising a carrier matrix formed of emulsified fruits and/or vegetables for administering probiotics and/or prebiotics to birds and small mammals. The dietary supplement would be suitable to help modify the microflora in the gastrointestinal tract of animals.

### EXAMPLE 16

A dietary supplement, according to the present invention, comprising a carrier matrix formed of emulsified skeletal tissue and/or internal organs of mammals, birds and/or fish for administering either enzymes or dried extracts of bovine or porcine pancreas containing enzymes to dogs and cats. Recommended dosages for enzyme activity would be 4,000-71,400 IU for lipase, 12,000-388,000 IU for protease and 12,000-460,000 IU for amylase per feeding. The dietary supplement would be suitable to help treat pancreatic insufficiency in dogs and cats.

### EXAMPLE 17

A dietary supplement, according to the present invention, comprising a carrier matrix formed of emulsified skeletal tissue and/or internal organs of mammals, birds and/or fish for administering fats and/or oils to dogs and cats, particularly, fats and/or oils having an omega-6 omega-3 fatty acid composition. Examples of an oil having an omega-6 omega-3 fatty acid composition include a variety of fish oils, canola oil, corn oil, sunflower oil, safflower oil, flax seed oil, borage oil, evening primrose oil, and combinations thereof. The recommended dosage would be 25-500 mg/kg body weight/day for omega-6 fatty acids and 10-250 mg/kg body weight/day for omega-3 fatty acids. The dietary supplement would be suitable as a treatment to help improve the skin and haircoat of both dogs and cats.

### EXAMPLE 18

A dietary supplement, according to the present invention, comprising a carrier matrix formed of emulsified apple and/or carrot for administering biotin to horses, mules, burros, and the like. The recommended level is 0.03-0.15 mg/kg body weight/day, a dose which has been proven to improve hoof health in horses and cows. The dietary supplement would be suitable as a treatment to help improve the hoof condition of horses, mules and burros.

### EXAMPLE 19

A dietary supplement, according to the present invention, comprising a carrier matrix formed of emulsified apple and/or carrot for administering phenylbutazone to horses, mules, burros, and the like. The recommended level is 2-8 mg/kg body weight/day, which is commonly used for pain relief. The supplement would be suitable as a treatment to help alleviate pain in horses, mules and burros.

### EXAMPLE 20

### Lyophilization

Example 20 provides one lyophilization process set forth by the International Society of Lyophilization—Freeze Drying. The invention should in no way be limited to this lyophilization process. Other lyophilization methods, again, will be readily ascertainable by those of ordinary skill in the art.

Concentration of active ingredient in process water: 5 wt. %-10 wt. %

Thermal Properties of the Formulation:

Degree of supercooling is 10° C.

Degree of crystallization is 1.

Eutectic temperature of −5.00° C.

Lyophilization Process:

Volume: 1 ml fill volume in a mold.

Freezing: The product would be loaded at a shelf temperature of 20° C. Once the product is completely loaded in the dryer, the shelf surface temperature would be reduced to −15° C. The shelf temperature would then be increased to −10° C., which would be the product temperature at which we plan to do our primary drying process.

Primary Drying: Just as soon as the shelf temperature reaches −10° C., we would begin to chill the condenser. By the time the condenser temperature reaches −40° C., all product temperatures would have reached and maintained −10° C. Since the formulation has a eutectic temperature of −5° C., there would be no mobile water present in the frozen matrix. Since the vapor pressure of ice at −10° C. is 260 kilopascal (1,950 mTorr), by maintaining a chamber pressure, by a nitrogen gas bleed, at 120 kilopascal (900 mTorr) and the shelf surface temperature of 20° C., the product temperature will remain at −10° C.±0.1° C. throughout the entire primary drying process.

Secondary Drying: The desired residual moisture would be obtained by reducing and maintaining the pressure, by means of a nitrogen gas bleed, in the chamber to 35 kilopascal (265 mTorr) and maintaining this pressure for one hour. This pressure would be sufficient to reduce the moisture to within the desired limits and at the same time be high enough to guarantee that there will be no backstreaming of hydrocarbon vapor from the vacuum pump.

Process Time: The entire lyophilization process would be less than approximately 8 hours.

I claim:

1. A method of administering a medicament to an animal, the method comprising:
forming a slurry comprising a natural substance;
mixing an effective amount of a medicament with the slurry to form a mixture;
pouring the mixture into a mold;
freezing the mixture to form a frozen mixture;
drying the frozen mixture to form a freeze-dried dietary supplement comprising the medicament; and
administering the dietary supplement to an animal.

2. The method as set forth in claim 1, wherein the animal is at least one of a small pet mammal, bird, companion mammal, and larger pet mammal.

3. The method as set forth in claim 1, wherein forming the slurry includes at least one of blending, grinding, emulsifying, homogenizing, and combinations thereof.

4. The method as set forth in claim 1, wherein the mixture has a eutectic point, and wherein freezing the mixture includes freezing the mixture below the eutectic point.

5. The method as set forth in claim 1, wherein drying the frozen mixture includes at least one of lyophilizing, freeze-drying, critical-point drying, and combinations thereof.

6. The method as set forth in claim 1, wherein the natural substance includes at least one of a fruit, vegetable, skeletal tissue, organ meat, connective tissue, cartilaginous tissue, and a combination thereof.

7. The method as set forth in claim 1, wherein the medicament comprises at least one of a nutrient, nutraceutical, medicine, enzyme, prebiotic, probiotic, Yucca schidigera, and a combination thereof.

8. The method as set forth in claim 1, wherein the mixture further comprises at least one of sugar, corn syrup, molasses, gum, humectant, starch, natural color, artificial color, natural flavor, artificial flavor, natural preservative, artificial preservative, xanthan, guar, karaya, locust bean, methylcellulose, carboxymethylcellulose, carageenan, agar, pectin, monoglyceride, diglyceride, polysorbate, sodium lauryl sulfate, sucrose ester, lecithin and a combination thereof.

9. The method as set forth in claim 1, wherein drying the frozen mixture includes drying the frozen mixture to a moisture content of less than about 28 wt %.

10. The method as set forth in claim 1, wherein drying the frozen mixture includes drying the frozen mixture to a moisture content of less than about 10 wt %.

11. The method as set forth in claim 1, wherein administering the dietary supplement to an animal includes administering at least 0.5 mg of medicament/kg body weight/day to dogs.

12. The method as set forth in claim 1, wherein administering the dietary supplement to an animal includes administering at least 0.1 mg of medicament/kg body weight/day to horses.

13. A method of preparing a dietary supplement comprising a medicament, the method comprising:
providing a natural substance to form a carrier matrix for the medicament;
slurrifying the natural substance to form a slurry;
mixing an effective amount of the medicament with the slurry to form a mixture;
pouring the mixture into a mold;
freezing the mixture; and
removing moisture from the mixture to form a shelf-stable dietary supplement.

14. The method as set forth in claim 13, wherein slurrifying includes at least one of emulsifying, grinding, blending, homogenizing, and combinations thereof.

15. The method as set forth in claim 13, wherein the mixture has a eutectic point, and wherein freezing the mixture includes freezing the mixture below the eutectic point.

16. The method as set forth in claim 13, wherein removing moisture includes at least one of evaporation, lyophilization, critical-point drying, freeze-drying, and combinations thereof.

17. The method as set forth in claim 13, wherein removing moisture includes drying the mixture to a moisture content of less than about 28 wt %.

18. The method as set forth in claim 13, wherein removing moisture includes drying the mixture to a moisture content of less than about 7 wt %.

19. The method as set forth in claim 13, wherein removing moisture includes drying the mixture to a moisture content of less than about 4 wt %.

20. The method as set forth in claim 13, wherein the natural substance includes at least one of a fruit, vegetable, skeletal tissue, organ meat, chondroprotective agent source, and a combination thereof.

21. The method as set forth in claim 13, wherein the mixture further comprises at least one of sugar, molasses, gum, humectant, starch, natural color, artificial color, natural flavor, artificial flavor, natural preservative, artificial preservative, xanthan, guar, karaya, locust bean, methylcellulose, carboxymethylcellulose, carageenan, agar, pectin, monoglyceride, diglyceride, polysorbate, sodium lauryl sulfate, sucrose ester, lecithin and a combination thereof.

22. The method as set forth in claim 13, wherein the medicament comprises at least one of a nutrient, nutraceutical, medicine, enzyme, prebiotic, probiotic, Yucca schidigera and a combination thereof.

23. The method as set forth in claim 13, wherein the medicament comprises at least one of protease, lipase, amylase, cellulase, lactase, inulin, dried chicory root, frucooligosaccharide, mannaoligosaccharide, soybean oligosaccharide, xylooligosaccharide, Lactobacillus species, Bifidobacterium species, Enterococcus species, Lactococcus species, Streptococcus thermophilus, Bacillus cereus, and a combination thereof.

24. The method as set forth in claim 13, wherein the medicament includes at least one of vitamin A, vitamin D, vitamin E, vitamin K, vitamin C, thiamin, riboflavin, pantothenic acid, niacin, pyridoxine, folic acid, biotin, vitamin B₁₂, choline, calcium, phosphorus, magnesium, sodium, potassium, chloride, zinc, iron, copper manganese, selenium, iodine, chromium, protein, amino acid, fats, fatty acid, and a combination thereof.

25. The method as set forth in claim 13, wherein the medicament comprises at least one of flavenoid, carotenoid, taurine, carnatine, glucosamine, chondroitin, conjugated linolcic acid, and a combination thereof.

26. The method as set forth in claim 13, wherein the medicament comprises at least one of ivermectin, moxidectin, fenbendazole, oxibendazole, oxifendazole, pyrantel pamoate, pyrantel tartrate, praziquantel, aspirin, phenylbutazone, mineral oil, petroleum jelly, and a combination thereof.

27. A freeze-dried dietary supplement for animals comprising:
a carrier matrix formed of a natural substance; and
an effective amount of a medicament, the medicament being intermixed with the carrier matrix to provide a chewable and palatable supplement.

28. The supplement as set forth in claim 27, wherein the natural substance comprises at least one of a fruit, vegetable, skeletal tissue, organ meat, connective tissue, cartilaginous tissue, and a combination thereof.

29. The supplement as set forth in claim 27, wherein the medicament comprises at least one of a nutrient, a nutraceutical, a medicine, enzyme, prebiotic, probiotic, Yucca schidigera, and a combination thereof.

30. The supplement as set forth in claim 27, further comprising at least one of sugar, corn syrup, molasses, gum, humectant, starch, natural color, artificial color, natural flavor, artificial flavor, natural preservative, artificial preservative, xanthan, guar, karaya, locust bean, methylcellulose, carboxymethylcellulose, carageenan, agar, pectin, monoglyceride, diglyceride, polysorbate, sodium lauryl sulfate, sucrose ester, lecithin and a combination thereof.

31. The supplement as set forth in claim 27, wherein the effective amount of the medicament is at least approximately 0.5 mg.

32. The supplement as set forth in claim 27, wherein the effective amount of the medicament is less than approximately 1000 mg.
